(19) United States
(12) Patent Application Publication
(10) Pub. No.: US 2014/0350411 A1
(43) Pub. Date: Nov. 27, 2014

(54) METHOD AND SYSTEM FOR DETECTING AND ALLEVIATING NECK DISCOMFORT OF A MOTOR VEHICLE OCCUPANT

(71) Applicant: Ford Global Technologies, LLC, Dearborn, MI (US)

(72) Inventors: Uwe GUSSEN, Huertgenwald (DE); Frederic STEFAN, Aachen NRW (DE); Christoph ARNDT, Moerlen (DE); Thomas RAMBOW, Aachen NRW (DE)

(73) Assignee: Ford Global Technologies, LLC, Dearborn, MI (US)

(21) Appl. No.: 14/272,107

(22) Filed: May 7, 2014

(30) Foreign Application Priority Data
May 23, 2013 (DE) 10 2013 209 601.1

(51) Int. Cl. A61F 7/00; A61N 5/06; G06K 9/00; A61B 5/11; B60N 2/48; B60N 2/56

(52) U.S. Cl. CPC A61F 7/0085; B60N 2/4876; B60N 2/5621; G06K 9/00335; G06K 9/00315; A61B 5/1116; A61N 5/0613; A61F 2007/0011; A61N 2005/0626; A61N 2005/0651; A61N 2005/0659
USPC 600/477; 607/107; 607/88

(57) ABSTRACT

A camera mounted in a motor vehicle cabin monitors an upper body and/or face of a vehicle occupant. A computer unit applies pattern recognition methods to compare an image from the camera with reference data stored in a database, and to determine based on the comparison that the occupant is experiencing neck discomfort. The neck discomfort determination is made based on the comparison indicating a change in occupant posture, a change in occupant facial expression, and/or a relaxation movement of the upper body and/or of a head of the occupant. If neck discomfort is detected, warm air and/or infrared light is directed toward the affected neck area to alleviate the discomfort.

### CROSS-REFERENCE TO RELATED APPLICATIONS

This application claims foreign priority benefits under 35 U.S.C. § 119(a)-(d) to DE 10 2013 209 601.1 filed May 23, 2013, which is hereby incorporated by reference in its entirety

### TECHNICAL FIELD

The present invention relates to a method and to a device for detecting and reacting to neck discomfort problems of occupants of a motor vehicle. The occupants may be the actual driver or else a front seat passenger or rear passengers.

### BACKGROUND

Despite the progress which has been made in the ergonomic design of vehicle seats and headrests, neck discomfort problems (such as neck stiffness, neck pain, or neck tension) is a relatively frequently occurring problem for motor vehicle users.

Such complaints occur, in particular, in a motor vehicle driver who frequently makes relatively long journeys. Effects which contribute to this are tense body posture, the relative immobility of the upper body and at the same time a need for relatively frequent head movements for the necessary observation of the events on the road as well as effects owing to drafts during the cooling operations of the air conditioning system or owing to the airstream when windows or a sunroof are opened.

The corresponding neck discomfort complaints which can be associated with muscle tension are not only unpleasant and troubling for the driver of the vehicle but can also adversely affect the driver's concentration capability or driving performance. It may be the case, for example, that the driver attempts as far as possible to avoid relatively large movements of his head (for example looking over his shoulder) due to his restricted mobility, resulting in his field of vision being restricted.

### SUMMARY

In a first disclosed embodiment, a method comprises operating a camera to monitor an upper body and/or a face of an occupant of a motor vehicle, applying pattern recognition methods to compare an image from the camera with reference data stored in a database, and determining based on the comparison that the occupant is experiencing neck discomfort.

In a further embodiment, a method comprises initiating a countermeasure to alleviate the neck discomfort.

The countermeasure may comprise directing warm air toward an affected neck area. The warm air may be directed by a nozzle arranged in a neck support or headrest of the vehicle. The warm air may be directed by a plurality of nozzles, individual nozzles or groups of nozzles of the plurality of nozzles being selectively activating to focus the warm air on the affected neck area. The warm air may be modulated in a pulsating fashion.

The countermeasure may comprise directing infrared light onto an affected neck area. The infrared light may be generated by a light emitting diode arranged in a neck support or headrest of the vehicle. The infrared light may be generated by a plurality of infrared light emitting diode matrix elements, the infrared light emitting diode matrix elements being used selectively to act in a focused fashion on the affected neck area.

In a further embodiment, the determination of the neck discomfort is made based on the comparison indicating at least one of a change in occupant posture, a change in occupant facial expression, and a relaxation movement of the upper body and/or a head of the occupant.

In a further disclosed embodiment, a system for a motor vehicle comprises a camera mounted in the motor vehicle and operative to monitor an upper body and/or a face of an occupant of the vehicle and a computer unit applying pattern recognition methods to compare an image from the camera with reference data stored in a database, and to determine based on the comparison that the occupant is experiencing neck discomfort.

Embodiments of the present invention described herein are recited with particularity in the appended claims. However, other features will become more apparent, and the embodiments may be best understood by referring to the following detailed description in conjunction with the accompanying drawings, in which:

### BRIEF DESCRIPTION OF THE DRAWINGS

FIG. 1 shows a schematic system diagram of an arrangement for carrying out a method according to the invention;

FIG. 2 shows a schematic illustration of a warm air application; and

FIG. 3 shows a schematic illustration of a heat radiation application.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

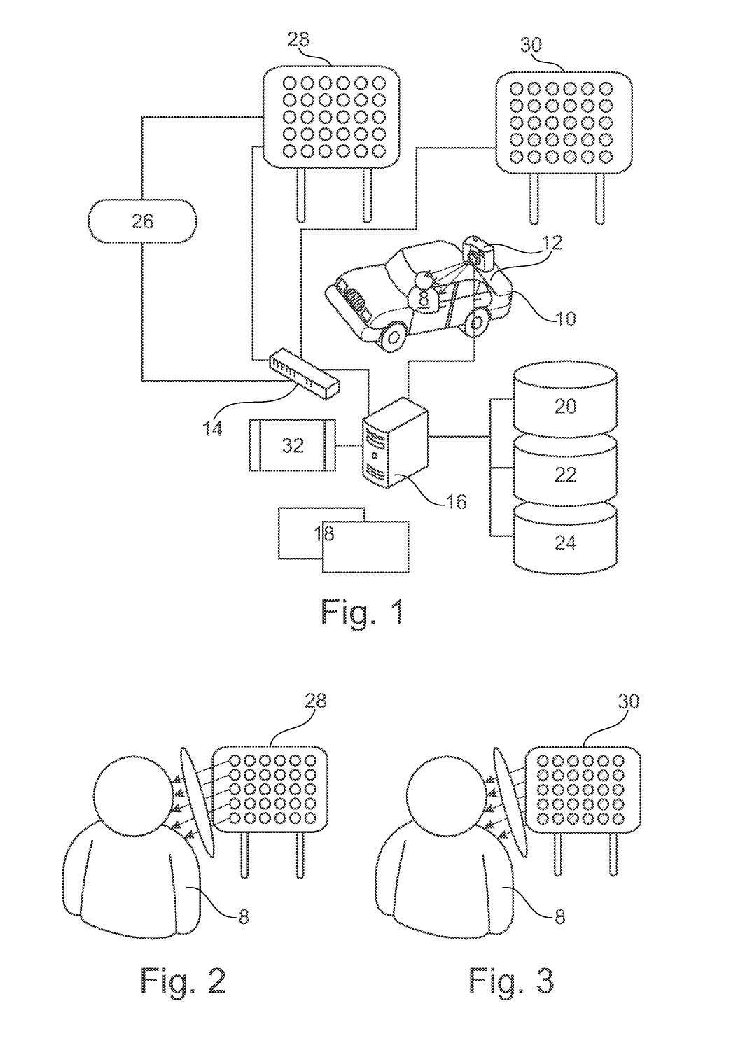

According to FIG. 1, the face and/or the neck and/or the upper body of a driver 8 (or if appropriate of a passenger) of a schematically illustrated motor vehicle 10 are/is monitored by means of at least one camera 12 arranged in the passenger compartment, wherein preferably more than one camera 12 is present in order to obtain images from various perspectives and therefore be able to evaluate movements of the driver in all three dimensions.

The image signals of the camera or cameras 12 are fed to a control unit 14 to which all sensors and actuators necessary within the scope of the invention are connected. The control unit 14 communicates for the evaluation of the images with a computing unit 16, wherein the corresponding units 14 and 16 can, of course, also be mounted together in one unit or combined with other motor vehicle control devices.

The image or plurality of images may be recorded over a predetermined time period. The image files may be buffered.

Within the scope of the invention, one or more images, if appropriate also moving images (videos), of the face and/or of the upper body region are recorded, these being preferably images which show the face and the upper body of the driver or other vehicle occupant. The described algorithms can be applied in each case also to occupants of the vehicle other than the driver, without this being mentioned each time here.

The computing unit 16 evaluates the images or videos and calculates certain standardized data (i.e. independent of size and stature) therefrom relating to the posture, the sitting position and the facial expression of the driver 8 or of the affected person within the scope of a pattern recognition algorithm 18 of the type well known in the art.

These data items are then compared with a database for correct sitting positions 20, and/or with a database for body postures and head postures 22 which indicate freedom from pain or which are associated with pain, and/or with a database for facial expressions 24 which indicate freedom from pain or which are associated with pain. By means of the pattern recognition methods it is detected whether the current images tend to indicate a neck discomfort problem in terms of various aspects, or tend to indicate that there is no sign of such a problem. On the basis of a point system, the control unit 14 then decides whether a potential neck discomfort problem is present or not. If it is, it is then possible, if appropriate, to take corresponding countermeasures after a dialogue with the driver 8.

The recorded images are preferably reduced, using pattern recognition methods of the type well known in the art, to specific features which are relevant for identification of neck discomfort problems, standardized (for example with respect to the size of the driver) and compared with stored data of typical persons with and/or without complaints related to neck discomfort. It is therefore possible to detect the general posture of the head, for example from the position of the face with respect to the upper body and/or to detect a rolling movement of the head, which represents the typical reaction to neck tension, by reference to the time profile of the position of the head.

In addition, the current sitting position can also be compared with a database for ergonomically favorable sitting positions, wherein the posture of the driver which is determined by means of image data can be taken into account and, if appropriate, a correction of the sitting position can then be proposed and, if appropriate, implemented directly by means of an automatic seat adjustment.

In addition, it is possible, under certain circumstances, to differentiate a normal facial expression from a tense facial expression by evaluating images by means of a neural network. A tense facial expression can be detected, for example, through furrowing of the brow, blinking in reaction to pain, distance between the eyes, etc.

The comparison data stored in at least one database can be provided at the factory. However, the data can also be modified by specifically recorded images of the driver in a state in which it is assumed that no neck discomfort problems are present, or in a state in which problems are suspected, in order to increase the recognition sharpness through learning processes.

As a result, the different criteria are processed according to a predefined weighting to preferably form a type of score. When a specific threshold value is exceeded or in the event of a significant deviation in an upward direction compared to an average value determined over a relatively long time, a possible neck discomfort problem is indicated.

Furthermore, by reference to the body posture and the facial expression of the person in question it is possible to determine, infer, or deduce where the problem is located (on the right or the left, for example), for which purpose, if appropriate, stored “historical” data from neck discomfort problems of this person which have already been detected earlier can also be used because corresponding vehicles are generally equipped at the same time with a person recognition system for taking into account individual vehicle settings.

As mentioned above, if the control unit 14 decides that a potential neck discomfort problem is indicated, it is then possible to take corresponding countermeasures for alleviating the problems after a dialogue with and/or confirmation by the affected person. The diagnosed or presumed neck discomfort problem may first be communicated to the driver or the affected person via a man/machine interface (for example a display or a voice announcement), if appropriate together with tips and instructions for exercises to alleviate the problems.

A possible countermeasure is the targeted use of warm air directed at the neck area of the affected person, i.e. allowing warm air to flow onto the affected neck area by means of at least one nozzle which may be arranged in the area of the headrest or neck support.

For example, if appropriate, pulsating warm air can be applied to a nozzle cluster 28 arranged in a neck support or headrest of the vehicle 10, via a warm air supply system 26. In this context, individual nozzles of the nozzle cluster 28 can be actuated by means of actuators in such a way that the warm air is concentrated or targeted on the affected regions and/or a certain massage effect is achieved. The ideal region of use of the warm air is controlled here by a detection unit for the best region 32 of use on the basis of the image data and/or on the basis of the driver interrogation and/or on the basis of “historic” information (stored last warm air applications for the corresponding person). The corresponding warm air application is illustrated more clearly once more in FIG. 2.

In one embodiment, a plurality of nozzles are provided in at least one nozzle cluster or nozzle matrix, and the stream of warm air is applied by selectively activating individual nozzles or nozzle groups in a way which is focused on the acutely affected neck area. Through this targeted application of warm air (for example on the right, on the left or in the center of the neck or further up or down) a particularly positive effect is achieved since sensory stimulation which is focused on a certain area generally has a more positive physiological affect than a nonspecific application of comparable stimulation over a wide area.

For this purpose, it may be necessary for the approximate location of the complaints as described above to be determined or to be communicated to the control system by the affected person. One possible way of finding this out independently of the described sensor system is for the various controllable warm air nozzles or warm air nozzle groups in the neck area to be activated cyclically and then to remain at a specific nozzle or a specific nozzle group in response to a signal by the affected person (for example voice controlled by the word “OK”).

In a further embodiment, a slight massaging effect can be achieved by a stream of warm air through pulsating modulation of the stream of warm air. The intensity of the pulsation and the pressure of warm air can, if appropriate, also be made adjustable.

In addition it is possible that a stream of warm air is activated preventively, if appropriate even without the presence of a neck problem, if severe cooling of the passenger compartment is currently being performed by the air conditioning system, in order to avoid problems caused by drafts.

A countermeasure which can be taken alternatively or additionally (at the same time or alternately with the warm air described above) in order to alleviate the problems is to heat the affected neck area by means of at least one infrared light emitting diode matrix which may be arranged in the area of the headrest or neck support.

Targeted application of heat to the affected areas can be carried out by means of one or more infrared light emitting diode clusters 30, wherein in turn the detection unit for the best region 32 of use permits targeted activation of individual light emitting diodes (cf. also FIG. 3). In this case, a plurality of infrared light emitting diode matrix elements, which may be approximately the size of a stamp, can also be provided, said infrared light emitting diode matrix elements can then be used selectively in order to act in a directed fashion on only the affected neck area in that heat patterns or even dot patterns are generated.

What has been stated above with respect to cycling the warm air nozzles also applies for the determination of the correct infrared light emitting diode matrix elements which are to be activated selectively in each case for the acute complaints, i.e. it is also possible to cycle, if appropriate, between the individual infrared heat sources until the affected person signals (verbally or otherwise) to stop the activation of the infrared heat sources in the current condition, or the location of the complaints can be determined automatically.

The alleviating measures described above can also be continued automatically after interruptions in a journey as soon as the corresponding person has been identified by the vehicle control system, until manual switching off by the affected person is carried out.

The abovementioned alleviating measures for neck discomfort can, if appropriate, also be activated manually by the driver or other affected occupant without automatic detection of a corresponding neck discomfort problem. Accordingly, it lies within the scope of the present invention to perform corresponding alleviating measures for neck discomfort problems as described above without preceding automatic detection.

As a result, the detection of a possible neck discomfort problem is performed purely optically by recording camera images, and therefore in a contactless fashion and in a way which is not at all invasive for the vehicle occupants.

Since in many driving assistance systems (at least in many driving assistance systems under development), at least one camera for observing the driver is provided, for example for detecting the degree of alertness of the driver, in many cases the present invention does not entail additional cost for hardware required for the detection of a possible neck discomfort problem.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method comprising:
operating a camera to monitor an upper body and/or a face of an occupant of a motor vehicle;
applying pattern recognition methods to compare an image from the camera with reference data stored in a database; and
determining based on the comparison that the occupant is experiencing neck discomfort.

2. The method of claim 1, further comprising:
initiating a countermeasure to alleviate the neck discomfort.

3. The method of claim 2, wherein the countermeasure comprises directing warm air toward an affected neck area.

4. The method of claim 3, wherein the warm air is directed by a nozzle arranged in a neck support or headrest of the vehicle.

5. The method of claim 3, wherein the warm air is directed by a plurality of nozzles, and wherein the warm air is focused on the affected neck area by selectively activating individual nozzles or groups of nozzles of the plurality of nozzles.

6. The method as claimed in claim 3, wherein the warm air is modulated in a pulsating fashion.

7. The method of claim 2, wherein the countermeasure comprises directing infrared light onto an affected neck area.

8. The method of claim 7, wherein the infrared light is generated by a light emitting diode arranged in a neck support or headrest of the vehicle.

9. The method of claim 7, wherein the infrared light is generated by a plurality of infrared light emitting diode matrix elements, the infrared light emitting diode matrix elements being used selectively to act in a focused fashion on the affected neck area.

10. The method of claim 1, wherein a plurality of images is recorded over a time period to form an image file and the image file is compared with the reference data.

11. The method of claim 1, wherein the determination of the neck discomfort is made based on the comparison indicating at least one of a change in occupant posture, a change in occupant facial expression, and a relaxation movement of the upper body and/or a head of the occupant.

12. A method comprising:
operating a camera to monitor an upper body and/or a face of an occupant of a motor vehicle;
applying pattern recognition methods to compare an image from the camera with reference data stored in a database;
determining based on the comparison that the occupant is experiencing neck discomfort; and
initiating a neck discomfort countermeasure comprising directing at least one of warm air and infrared light toward a neck area of the occupant.

13. The method of claim 12, wherein the warm air is directed by a nozzle arranged in a neck support or headrest of the vehicle.

14. The method of claim 12, wherein the warm air is directed by a plurality of nozzles, individual nozzles or groups of nozzles of the plurality of nozzles being selectively activating to focus the warm air on the neck area.

15. The method of claim 12, wherein the infrared light is generated by a light emitting diode arranged in a neck support or headrest of the vehicle.

16. The method of claim 12, wherein the infrared light is generated by a plurality of infrared light emitting diode elements, the infrared light emitting diode elements being used selectively to act in a focused fashion on the neck area.

17. The method of claim 12, wherein the determination of the neck discomfort is made based on the comparison indicating at least one of a change in occupant posture, a change in occupant facial expression, and a relaxation movement of the upper body and/or of a head of the occupant.

18. A system comprising:
a camera mounted in a motor vehicle and monitoring an upper body area and/or a face of an occupant of the vehicle; and
a computing unit applying pattern recognition methods to compare an image from the camera with reference data stored in a database, and to determine based on the comparison that the occupant is experiencing neck discomfort.

19. The system of claim 18 further comprising:
a nozzle mounted in the vehicle adjacent to a headrest and activated by the computing unit to direct warm air toward a neck area of the occupant.

20. The system of claim 18 further comprising:
an infrared light source mounted in the vehicle adjacent to a headrest and activated by the computing unit to direct infrared light toward a neck area of the occupant.
